(19) United States
(12) Plant Patent Application Publication
(10) Pub. No.: US 2010/0319095 P1
(43) Pub. Date: Dec. 16, 2010

(54) Nectarine tree denominated 'summer lion l'

(50) ;

(76) Inventor: Donald M. Serimian, Selma, CA (US)

Correspondence Address: Rodney K. Worrel;Worrel & Worrel, St. Croix Professional Center, 2109 W. Bullard Avenue, Suite 121, Fresno, CA, 93711-1258, US

(21) Appl. No.: 12/456,159

(22) Filed: Jun. 15, 2009

(51) Int. Cl. A01H 5/00

(52) U.S. Cl. PLT/190

(57) ABSTRACT

A new and distinct variety of nectarine tree which is somewhat similar to ‘Summer Lion II’, but from which it is distinguished by producing fruit which are mature for harvesting and shipment approximately seven days before ‘Summer Lion II’ fruit in the San Joaquin Valley of central California, and wherein the fruit has, good flavor, very large size with dark red coloration over most of the fruit surface.

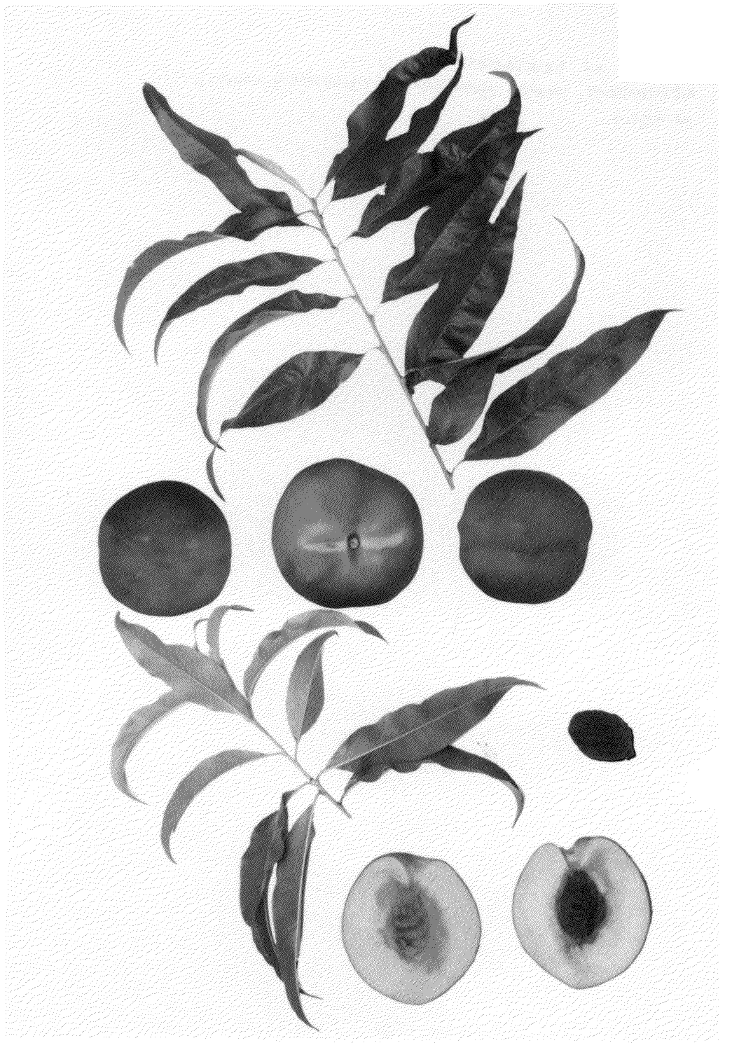

### CROSS-REFRENCE TO RELATED APPLICATIONS

Not applicable.

### STATEMENT REGARDING FEDERALLY SPONSERED RESEARCH OR DEVELOPMENT

Not applicable.

### LATIN NAME OF THE GENUS AND SPECIES OF THE PLANT CLAIMED

Prunus Persica Var Nectarina

### SPECIFICATION

### Background of the New Variety

The present invention relates to a new and distinct variety of nectarine tree, Prunus perisica var nectarina, which will hereinafter be denominated varietally as the ‘Summer Lion I’ nectarine tree and more particularly, to a nectarine tree which produces red to dark red blush color fruit with yellow orange flesh color of large size which matures for commercial harvesting and shipment approximately July 1^{st}-July 15^{th}, the commercial harvesting being about one week before Summer Lion II (U.S. Plant Pat. No. 6,455) in the San Joaquin Valley of central California.

The development of new varieties of plant life requires rigorous effort, including, among others, acute observation to recognize possible new varieties; a scientific knowledge of botanical data and genetic identifiers to arm the individual with a sufficient knowledge to be aware of what may be new characteristics in genus and species which are otherwise known; knowledge of a plethora of variable attributes and, conversely undesirable characteristics which may well control whether a new variety is a success; the reasons for such attractive and unattractive characteristics may be, for example, the bright color of the fruit which may be attractive to customers or, conversely unattractive characteristics such as, a susceptibility to diseases; good fortune in discovering a good variety either where produced by natural selection or a hybridization program; or any of a multitude of other considerations.

Given this seemingly unending list of variables, it has been observed that it is miraculous that any but mundane varieties are commercialized so as to bring them to market.

The variety of nectarine tree of the present invention results from all of the foregoing knowledge, powers of observation, experience a lifetime of effort of the invention these and others in a development program which has produced many superior varieties of fruit.

With this background in mind, the subject inventor hereof believes the instant variety to be a very promising candidate for commercial success, particularly in view of filing a gap in the commercial growing season and in its attractive appearance which greatly enhances the likelihood of commercial success.

### ORIGIN AND SEXUAL REPRODUCTION OF THE NEW VARIETY

The present variety of nectarine tree was discovered by the inventor in the spring of 2001 and was, the result of cross-pollination between the ‘June Lion’ nectarine tree (U.S. Plant Pat. No. 13,792) and the ‘Summer Lion II’ nectarine tree (U.S. Plant Pat. No. 6,544). In the spring of 2003 budwood was selected from one tree, grafted, on Nemaguard rootstock in the inventor's block of a ‘Summer Lion II’ nectarine orchard. Over the past four years, the inventor has observed that in six of these nectarines trees, the fruit matured one week earlier and were significantly larger than the known varieties. The inventor has observed the asexually reproduced trees of the instant variety and found that in all respects the resulting progeny are identical to that of the original tree of new variety.

### SUMMARY OF THE NEW VARIETY

The nectarine tree of the new variety is characterized by producing fruit which is ripe for commercial harvest and shipment approximately July 1^{st}-July 15^{th} in the San Joaquin Valley of central California, about seven days earlier than ‘Summer Lion II’ nectarine tree. The new variety of the present invention differs from its parents primarily in date of ripening, a darker red color, and larger fruit.

### BRIEF DESCRIPTION OF THE DRAWING

The drawing is a colored photograph displaying representative portions of the new variety of nectarine tree hereof with a terminal shoot showing the upper surface of the leaves at the top of the photograph; fruit of the new variety, the fruit on the left displaying the stem end of the fruit; the fruit in the middle displaying the blossom end of the fruit; and the fruit on the right displaying the side view and suture of the fruit; another terminal shoot displaying the bottom surface of the leaves; a stone; and, at the bottom of the photograph, one of the fruit sectioned and laid open, the half on the left displaying the pit well and flesh thereof and the half on the right displaying the stone in the pit well along with the flesh of the fruit.

### DETAILED DESCRIPTION

Referring more specifically to the botanical details of the new variety of nectarine tree, the following has been observed under the ecological conditions prevailing in the orchard of origin in an experimental block near Selma, Calif. in the San Joaquin Valley of central California. All major color designation are by reference to the Dictionary of Color, by Maerz and Paul, First Edition, 1930. Common color names are also occasionally employed.
Tree:
   Size.—Approximately, 426.72 cm to 457.2 cm (14 to 15 feet) high with a width approximately spreading to 335.28 cm to 365.76 cm (11 to 12 feet.)
   Vigor.—Very good with approximately 76.2 cm to 91.44 cm (2½ to 3 feet) of new growth.
   Figure.—Upright and spreading.
   Productivity.—Very good to excellent for 5^{th} leaf.
   Regularity of bearing.—Regular.
Trunk:
   Size.—73.66 cm (29 inches) in circumference, 84.00 cm (7 inches) above graft.
   Surface.—Rough.
   Color.—P1.7 C8— Mauve Taupe Copra.
   Lenticels.—Sparse, on trunk, lenticular in shape, approximately 3 mm (½ inch) long and 1 mm (0.04 inch wide).
Branch:
   Size.—20.955 cm (8¼ inches) in circumference, 38.1 cm (15 inches) above crotch angle, angle of branches approx. 58°.
   Surface texture.—Mature — moderately smooth; Immature — Smooth.
   Color code.—Mature Branches — from Pl. 7 C8 Mauve Taupe Copra to Pl. 7 C9 Chicle Sarouk+. Immature Branches — Pl. 18 J7 Spring gr.
   Lenticels.—Many — 20 over a 10.16 cm (4 inch) space.
Leaves:
   Size.—Length — 137 to 165 mm (5.37 to 6.50 inch). Width — 37 to 46 mm (1.46 to 1.81 inch). Shape — Lanceolate.
   Color code.—Upwardly disposed surface Pl. 23 J8 Cossack green. Downwardly disposed surface — Pl. 22 L10 Brunswick gr. Lt.
   Marginal form.—Crenate.
   Leaf margin.—Slightly undulate.
   Leaf midvein color.—Pl. 18 J7 Spring gr. Thickness — 1 to 1½ mm (0.04 to 0.06 inch).
   Glandular characteristics.—1 to 3 at base at leaf. Shape — Reniform. Color — Pl. 20 L9 Eden gr.
   Petiole.—Length — 8 to 15 mm (0.31 to 0.59 inch) Thickness — 2 mm (0.078 inch). Color — Pl. 19 K8 Near Bud gr.
   Stipule.—On younger growing tips, small 2 mm (0.079 inch). Color — P1. 17 L7 Viridine gr.
Flowers:
   Generally.—The following description was developed on Mar. 11, 2008.
   Date of bloom.—75% to 95% on Mar. 11, 2008, average for nectarine cultivars in the same fruit growing areas.
   Flower bud.—Hardy for growing conditions in San Joaquin Valley of central California.
   Flower buds.—Size — 5 mm (0.21 inch) to 7 mm (0.28 inch) in diameter. Length — 4 mm (0.019 inch) to 7 mm (0.28 inch) Form — Conic. Closely attached to bud sticks. Generally large in size and showy.
   Bloom quantity.—Moderately dense in one year old wood, flower buds 2 to 3 per node.
   Flower diameters.—37 mm (1.46 inch) to 45 mm (1.77 inch).
   Petals.—Size — Generally large. Length — 19 mm (0.075 inch) to 20 mm (0.79 inch). Width — 20 mm (0.79 inch) to 21 mm (0.83 inch). Form — Broadly ovate.
   Petal claw.—Form — Truncate. Size — Short. Length — 2 mm (0.081 inch) Width — 1.5 mm (0.06 inch)
   Petal margins.—Slightly cupped and moderately undulate.
   Petal apex.—Rounded.
   Pedicel.—Size — Short. Length — 2 mm (0.08 inch). Thickness — Average 2.5 mm (0.1 inch).
   Calyx.—External Surface — Glabrous and somewhat veined. Color — Variable mature calyx Pl. 7 E5 — Mauverose, base Pl. 9 L7 Cavalry deep chrome 4.
   Sepals.—Surface — Pubescent. Size — Length — 6 mm (0.24 inch) to 7 mm (0.28 inch). Width — 5 mm (0.21 inch). Form — Conic. External Color — Pl. 7 J7 Roan.
   Anthers.—Plump. Size — Average Color — Ventral — Pl. 5 L12 Chrysanthum. Dorsal — Pl. 11 J4 Mustard.
   Stamens.—Slightly shorter than the pistil and variable in length 9 mm (0.36 inch) to 15 mm (0.06 inch) (35-40 in number).
   Filament color.—Pl. 1 J4 Cherry blossom.
   Pistil size.—Length average 22 mm (0.87 inch).
   Style.—Color — Pl. 17 H2 Light yellow gr. Surface — Glabrous.
   Ovary color.—Pl. 17 L8 Neva gr.
Fruit:
   Maturity.—July 1^{st}-July 15^{th} in the San Joaquin Valley of central California.
   Size.—Large — Borne in clusters of two or single — weight 12 to 15 oz. (340 to 425 grams) Diameter in the axial plane — 74 to 83 mm (2.91 to 3.27 inch). Transverse in the suture plane — 71 to 82 mm (2.80 to 3.23 inch). Transverse at right angles to suture plane — 70 to 78 mm (2.76 to 3.07 inch).
   Form.—Uniformity — Good. Symmetrical or Asymmetrical — Symmetrical. Suture — Somewhat shallow on basal end, very shallow on apical end. Ventral Surface — Generally smooth. Stem Cavity — Average in size — Often a line of ground yellow color Pl. 10K3. Width — 20 to 28 mm (0.79 to 1.10 inch). Depth — 15 to 17 mm (0.59 to 0.67 inch). Length — 25 to 32 mm (0.98 to 1.26 inch). Shape — Oval.
   Stem.—Short — 8 mm (8.31 inch). Caliper — 3.97 mm (0.156 inch).
   Apex.—Slightly raised.
   Pistil point.—Oblique.
Skin:
   Thickness.—Average for nectarine.
   Texture.—Fully glabrous.
   Tendency to crack.—None observed.
   Color code.—Blush Color — Pl. 5 D1 Brickdust Red to Pl. 8 L5 Zanzibar. Ground Color — limited area of surface with less than 10% — Pl. 10 12 Aurelia yellow. Flesh Color — Pl. 4 LA Sunflower Dandelion Skin with RedFlecks — Pl. 4 H10 Holly Berry. Color of Surface of Pit Cavity — Over ⅔rds P1.4 L10 Holly Berry. Color of Pit Well — Pl. 10 L4 Holly Berry.
   Juice production.—Very good.
   Flavor.—Very good.
   Aroma.—Aromatic.
   Fibers.—Number — Few/Texture — Firm.
   Ripening.—Evenly.
   Eating quality.—Excellent.
Stone:
   Free or cling stone.—Semi clingstone.
   Fibers.—Number — Few Length — 5 to 10 mm (0.20 to 0.39 inch).
   Size.—Length — 40 mm (1.57 inch). Width — 30 to 32 mm (1.18 to 1.26 inch). Thickness — Average 23 mm (0.91 inch).
   Form.—Oblong to ovate.
   Apex shape.—Acute with pointed tip.
   Color dry.—Pl. J5 L123 Vinyard Oporto +.
   Base.—Flat with slanted sides.
   Hilium.—Ovate with collar — 10 mm; long, 3 mm.
   Ridges.—Very wide at stem (base/end), dorsal edge tightly grooved, ventral edge wider.
   Tendency to split.—Small number observed.
   Use.—Fresh Market.
Keeping and shipping quality — Very good.
Resistance to disease: None known.
Harvesting and shipping — July 1-July 15.
Has the New Variety Been Sold — No.
Has the New Variety Been Publicly Used of Exhibited — No.
Has Any Reproducible Parts of Plant Been Given Away — No.
Name of the New Variety — ‘Summer Lion I’. Although the new variety of nectarine tree has the described horticulture characteristics indicated above that are growing conditions prevailing near the town of Selma, in the San Joaquin Valley of central California, it is to be understood that variations of the usual magnitude may occur due to changes in growing conditions such as irrigation, fertilization, pruning, pest and disease control as well as climatic variations.

Having thus described and illustrated our new variety nectarine tree which we claim as new and desire to be secured by Plant Patent:

1. A new and distinct variety of nectarine tree substantially as described and illustrated which is somewhat similar to ‘Summer Lion II’ but from which it is distinguished by producing fruit which are mature for commercial harvesting and shipment approximately seven days before ‘Summer Lion II’ nectarine tree in the San Joaquin Valley of central California, and which are very good size with distinct dark red color over its skin surface.
